(19) United States
(12) Patent Application Publication
(10) Pub. No.: US 2012/0053485 A1
(43) Pub. Date: Mar. 1, 2012

(54) Catheter Having Needle And Expandable Support Member And Methods Of Use

(75) Inventor: Eliot Frank BLOOM, Hopkinton, NH (US)

(73) Assignee: Salient Surgical Technologies, Inc., Portsmouth, NH (US)

(21) Appl. No.: 12/873,977

(22) Filed: Sep. 1, 2010

(51) Int. Cl. A61B 1/267; A61M 25/10; A61B 17/34; A61B 10/02; A61B 18/18

(52) U.S. Cl. 600/567; 606/33; 606/185; 600/104

(57) ABSTRACT

A catheter includes a catheter shaft, an expandable member coupled to an outer surface of the catheter shaft, and a needle having a distal end portion disposed on an outer side of the catheter shaft adjacent the expandable member. The expandable member is selectively radially expandable from the radial perimeter of the catheter for making contact with a body lumen wall. The needle is configured to extend laterally from the outer side of the catheter shaft at a non-zero angle relative to the longitudinal axis of the catheter shaft. The needle contacts the expandable member during the selective radial expansion of the expandable member such that the non-zero angle of the extended needle is controlled by the expansion of the expandable member. The radial center of the expandable member can be eccentrically arranged with respect to a longitudinal axis of the catheter. The expandable member can include at least one opening for allowing bodily fluid or gas (e.g., air) to pass through when the expandable member is expanded in a body lumen.

### BACKGROUND OF THE INVENTION

1. Field of the Invention

The present invention relates to medical devices, and in particular to endoscopes and other devices for gaining access to inner organs or other tissue for bringing a medical tool such as biopsy and ablation needles to a target for diagnostic and therapeutic purposes, such as bronchoscopes and other devices for gaining access to the pulmonary airway.

2. Background Art

In general, catheters are tubes that can be inserted into a body lumen to remove or inject fluids, or bring a medical tool to a remote target tissue for diagnostic and therapeutic purposes. For example, catheters can be delivered percutaneously to a target tissue, such as by being led over a guidewire inserted through an incision in the skin and through a body lumen to the target tissue. Catheters can include endoscopic devices to allow the physician to examine the hollow organ or cavity were the distal end of the catheter is located. The catheter can include a medical tool, such as a biopsy needle for diagnosis of the target, such as for the diagnosis of abnormal tissues. For treatment of the target, the catheter can include a needle for local delivery of a medicinal substance to the target tissue. For the ablation of cancerous tumors, for example, the catheter can include an ablation needle for radiofrequency (RF) ablation treatment of the target tissue. For such diagnostic and therapeutic procedures, it remains a challenge not only to bring the catheter to the target with minimal discomfort and risk to the patient, but also to then deploy the needle with sufficient accuracy so as to engage the target tissue without multiple attempts, which can endanger the patient and complicate the procedure. In addition, in some procedures, it undesirable, and can be life-threatening, to completely block the body lumen with the catheter, and therefore it also remains a challenge to stabilize the distal end of the catheter in the body lumen in a manner that avoids occlusion of the lumen and stopping blood flow, or other bodily fluids or gases, through the lumen, such as avoiding occlusion of the airways and stopping airflow when the procedure involves catheterization of the lungs.

For example, the lungs deliver oxygen to the body by directing air through numerous air passageways that lead from the trachea to respiratory bronchioles to alveolar sacs. Bronchoscopy provides clinically useful information by direct inspection of the pulmonary airway, particularly the inspection of branches of the tracheobronchial tree that forms the airways that supply air to the lungs. Bronchoscopes come in both flexible and rigid forms and are structured so as to allow the passage of air past the bronchoscope when it is inserted in the body. Referring to FIG. 1 which depicts a pair human lungs 10 and the tracheobronchial tree, airflow in lungs 10 generally follows a path from the trachea 12, through the main (or primary) left and right branch bronchial tubes 14, then through the sub-bronchial tubes 16. Though not shown, the sub-bronchial tube 16 lead to the numerous tiny bronchioles (not shown) that lead to alveolar sacs (not shown) which include multiple alveoli separated by alveolar walls for the exchange of oxygen and carbon dioxide.

Bronchoscopes usually contain a vision component (e.g. an optical component, such as fiberoptics) that transmits an image from the distal tip of the instrument to a display monitor to allow the physician to view the airways. A rigid bronchoscope is usually used with general anesthesia, and typically incorporates a straight, stainless-steel hollow tube of varying length and diameter. A rigid bronchoscope has a larger channel than a flexible bronchoscope which permits the rigid bronchoscope to be used for removing secretions, blood, or foreign objects lodged in the airway, as well as for removing bulky tumors or large tissue samples for biopsy, introducing of radioactive materials, and placing stents. A flexible bronchoscope is longer and thinner than a rigid bronchoscope. The flexible bronchoscope usually includes a channel for suctioning or instrumentation, but such channel is significantly smaller than that in a rigid bronchoscope. A specimen from the trachea, main bronchial tubes 14, or sub-bronchial tubes 16 can be retrieved using a flexible bronchoscope by means of brush biopsy, bronchoalveolar lavage, or biopsy forceps or needle. The flexible bronchoscope is generally used with local anesthesia and sedation, is usually more comfortable for the patient, and offers a better view of the smaller airways than a rigid bronchoscope.

Conventional practices using flexible or rigid bronchoscopes for biopsy of the trachea or bronchi (e.g., biopsy of the lymph nodes located around the trachea and the primary bronchi) include delivery of a biopsy needle to the trachea or bronchi wall. For example, biopsy of lymph nodes of the bronchi typically requires introduction of a bronchoscope through the nose or mouth and extending the bronchoscope through the trachea to the lymph nodes located around the trachea and the primary left and right bronchi. A biopsy needle is extended from the distal end of the bronchoscope to the lymph node tissue. However, conventional practices do not ensure accurate delivery of the needle to the targeted lymph node area, since conventional bronchoscopes are not stabilized in the airways of the patient but rather simply extend through the airways with space for the physician to manipulate the bronchoscope and for the patient to breathe. In addition, conventional bronchoscopes do not stabilize the biopsy needle when extended, and there is no guarantee that the biopsy needle will contact the target tissue (e.g., bronchi wall) or that the needle will be oriented at the optimal angle to the target tissue (e.g., 90 degrees to the bronchi wall). While the location of the bronchoscope in the tracheobronchial tree can be known by use of a location markers on the bronchoscope, the uncertainty in the radial position and orientation angle of the needle upon delivery can raise the risk of the biopsy procedure. For example, the biopsy needle can accidentally be extended through the trachea wall and puncture the nearby aorta. In some cases, multiple attempts of delivering the biopsy needle to the bronchi wall are required to allow the target tissue to be sampled. Multiple needle attacks on tissue can be undesirable, since each needle attack can increase the risk of an adverse bodily reaction (e.g., excessive bleeding) at the needle contact site. Moreover, the targeted tissue may be such that single or multiple needle attacks at or near the target are possible during one procedure, but multiple subsequent needle attacks on the same area would be unsuccessful.

There is a continuing need for catheters that can deploy needles to targeted tissue for diagnosis and therapy, in which the catheter can be held securely and accurately in place, and in which the orientation angle of the needle contacting the target tissue can be controlled, and a further need to do so without restricting airflow or flow of bodily fluids and with or without a vision component.

### BRIEF SUMMARY OF THE INVENTION

The present invention satisfies the above needs by providing catheters that have one or more expandable support members that can hold the catheter in the body lumen, without restricting airflow or flow of bodily fluids through the lumen, and by providing catheters that have a needle that can contact the targeted tissue at a predetermined and/or adjustable angle, and can do so without requiring any form of vision assistance. The present invention provides further related advantages, as will be made apparent by the description of the embodiments that follow.

Catheters having a needle and one or more expandable support members are presented. In some embodiments, a catheter includes an elongated catheter shaft, an expandable member, and a needle. The catheter shaft has a proximal end portion, a distal end portion, a radial perimeter, and a longitudinal axis. The expandable member is coupled to an outer surface of the distal end portion of the catheter shaft. The expandable member extends around the radial perimeter of the catheter shaft and is selectively radially expandable from the radial perimeter of the catheter shaft for making contact with a body lumen wall. The needle has a distal end adjacent the expandable member and disposed on an outer side of the distal end portion of the catheter shaft. The distal end of the needle is configured to extend laterally from the outer side of the catheter shaft at a non-zero angle relative to the longitudinal axis of the catheter shaft. The expandable member contacts the needle during the selective radial expansion of the expandable member such that the non-zero angle of the extended needle is controlled by the expansion of the expandable member.

Methods of using the catheter are also presented. In some embodiments, a catheterization method for maneuvering a needle to contact a targeted tissue, includes introducing the catheter into a body lumen, positioning the distal end portion of the catheter shaft so that the distal end of the needle faces opposite a target location on the body lumen wall; and expanding the expandable member in the body lumen to contact the body lumen wall. The expandable member contacts the needle during expansion and the expandable member moves the needle to laterally extend to a non-zero angle relative to the longitudinal axis of the catheter shaft.

The catheter can be a bronchoscope. In some embodiments, a bronchoscope includes an elongated tubular shaft, a needle, and a non-inflatable expandable member. The tubular shaft has a proximal end portion, a distal end portion, a radial perimeter, and a longitudinal axis. The tubular shaft also has a lumen, a needle port disposed at the distal end portion of shaft, and a ramp. The shaft defines the lumen which communicates with the needle port. The ramp extends from a distal side of the needle port into the lumen. The needle has a distal end portion and is slidably disposed in the lumen such that the needle has a retracted configuration and an extended configuration. In the extended configuration, the distal end portion of the needle contacts the ramp and extends laterally through the needle port at a non-zero angle relative to the longitudinal axis of the shaft. The non-inflatable expandable member is coupled to an outer surface of the distal end portion of the shaft. The expandable member has a proximal skirt, a distal skirt longitudinally spaced from the proximal skirt, and a plurality of elongated struts extending from the proximal skirt to the distal skirt and extending around the radial perimeter of the shaft. One of the proximal and distal skirts is fixedly mounted on the shaft, and the other of the distal and proximal skirts is slidably mounted on the shaft such that the expandable member has a retracted position and expanded position. In the expanded position, the plurality of elongated struts radially expand from the radial perimeter of the shaft for making contact with a lumen wall of an airway of a tracheobronchial tree. The plurality of struts splay apart away from each other when the expandable member is moved from the retracted position to the expanded position. An opening is provided between each of the splayed apart struts for allowing air to pass through when the expandable member is in the expanded position in the airway.

### BRIEF DESCRIPTION OF THE DRAWINGS/FIGURES

The accompanying drawings, which are incorporated herein and form a part of the specification, illustrate the present invention and, together with the description, further serve to explain the principles of the invention and to enable a person skilled in the pertinent art to make and use the invention.

FIG. 1 is a schematic illustration of a pair of lungs showing the tracheobronchial tree.

FIG. 2 is a side view of a portion of a catheter according to an embodiment presented herein, showing an outer delivery catheter.

FIG. 3 is a side view of a distal end portion of the catheter of FIG. 2, showing the delivery catheter retracted to expose an inner catheter including a catheter shaft and a needle, according to an embodiment presented herein.

FIG. 4 is a side view of the catheter of FIG. 3, showing a proximal balloon support member in an inflated, expanded position, according to an embodiment presented herein.

FIG. 5 is a side view of the catheter of FIG. 4, showing a distal balloon support member in an inflated, expanded position, according to an embodiment presented herein.

FIG. 6 is a side view of the catheter of FIG. 5 according to an embodiment presented herein, in which the balloon support members are concentric with the longitudinal axis of the catheter shaft.

FIG. 7 is a side view of the catheter of FIG. 5 according to an embodiment presented herein, in which the balloon support members are eccentric with the longitudinal axis of the catheter shaft.

FIG. 8 is a side view of the catheter of FIG. 5 located in a left main bronchus, according to an embodiment presented herein.

FIG. 9 is a perspective view of a distal end portion of an exemplary radiofrequency ablation needle according to an embodiment presented herein.

FIG. 10 is a side view of a distal end portion of a catheter according to an embodiment presented herein, in which two balloon support members sandwich a cannula, and showing the balloon support members in a deflated, retracted position.

FIG. 11 is a side view of the catheter of FIG. 10, showing the balloon support members in an inflated position, and a needle extending from the cannula at about 90 degrees to the longitudinal axis of a catheter shaft, according to an embodiment presented herein.

FIG. 12 is a side view of the catheter of FIG. 10, showing the balloon support members in another inflated position, and the needle extending from the cannula at about 60 degrees to the longitudinal axis of the catheter shaft, according to an embodiment presented herein.

FIG. 13 is a bottom view of a distal end portion of the catheter of FIG. 10, showing the balloon support members in an inflated position, with the needle retracted inside the cannula.

FIG. 14 is a bottom view of a distal end portion of the catheter of FIG. 13, showing the needle extending from the cannula.

FIG. 15 is a schematic view of the cannula with the needle retracted therein.

FIG. 16 is a side view of a distal end portion of a bronchoscope including a bronchoscope shaft and a non-inflatable expandable support member in a retracted position, according to an embodiment presented herein.

FIG. 17 is a side view of the catheter of FIG. 16, showing the expandable support member in an expanded position.

FIG. 18 is a side view of the catheter of FIG. 17, showing a needle extending through a needle port in the bronchoscope shaft.

FIG. 19 is a schematic view of the needle retracted within the lumen of the bronchoscope shaft.

FIG. 20 is a schematic view of the needle extending through the needle port in the bronchoscope shaft.

FIG. 21 is a side view of the bronchoscope of FIG. 20 located in a trachea and including a second support member, according to an embodiment presented herein.

FIG. 22 is a side view of a distal end portion of a bronchoscope including a non-inflatable expandable support member in a retracted position within a trachea, according to an embodiment presented herein.

FIG. 23 is a side view of the bronchoscope of FIG. 22, showing the support member in an expanded position within the trachea, according to an embodiment presented herein.

FIG. 24 is an axial view of the bronchoscope of FIG. 23, according to an embodiment presented herein.

### DETAILED DESCRIPTION OF THE INVENTION

Unless defined otherwise, all technical and scientific terms used herein have the same meaning as commonly understood by one of ordinary skill in the art to which this invention belongs. In case of conflict, the present application including the definitions will control. Also, unless otherwise required by context, singular terms shall include pluralities and plural terms shall include the singular. All publications, patents and other references mentioned herein are incorporated by reference in their entireties for all purposes.

The term “invention” or “present invention” as used herein is a non-limiting term and is not intended to refer to any single embodiment of the particular invention but encompasses all possible embodiments as described in the application.

As used herein, the term “catheter” generally refers to a tube for insertion into a body lumen, and can be either flexible or rigid. For example, a catheter can include a flexible or rigid bronchoscope shaft. In the description of the exemplary embodiments that follow, catheters according to the present invention will be described as being a flexible or rigid bronchoscope device; however, it should be understood that the present invention is not limited to bronchoscopes, but that modifications and variations of the embodiments described herein may be made for different catheter applications. For example, other embodiments of catheters in accordance with the present invention are possible, including by way of example and not limitation, catheters configured for cardiac catheterization, catheterization of the hepatic artery, urinary catheterization, and colonoscopy, or other applications involving catheterization via natural orifices or minimally-invasive procedures (e.g., neurocatheterization and single-incision laparoscopic procedures).

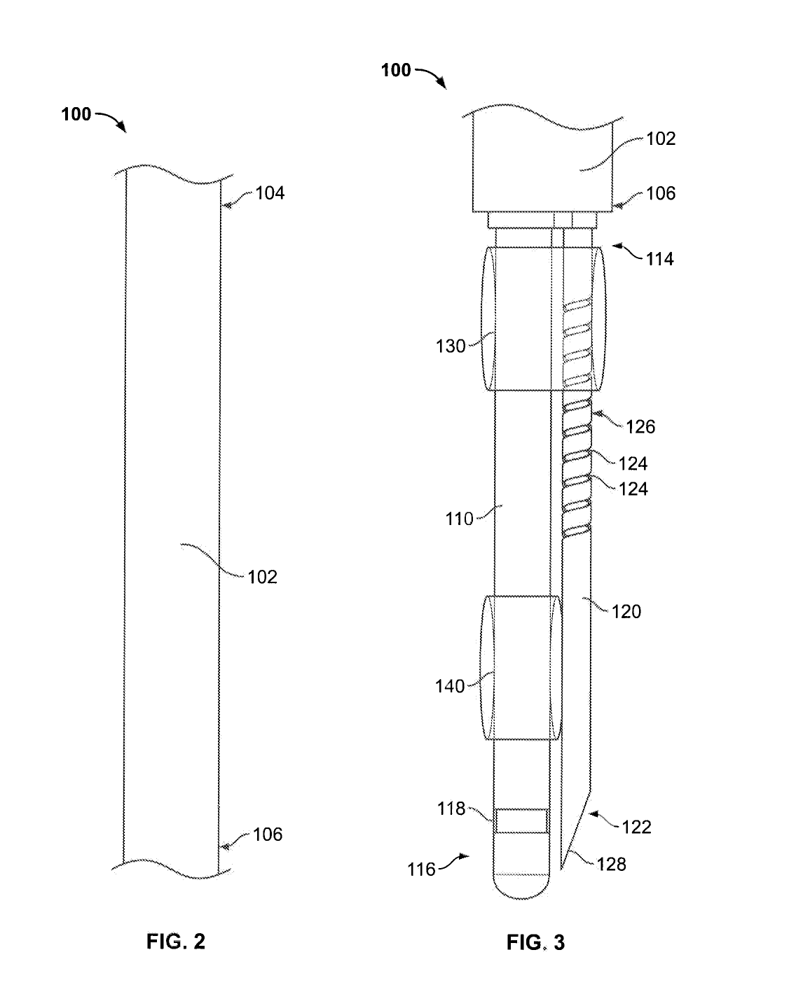

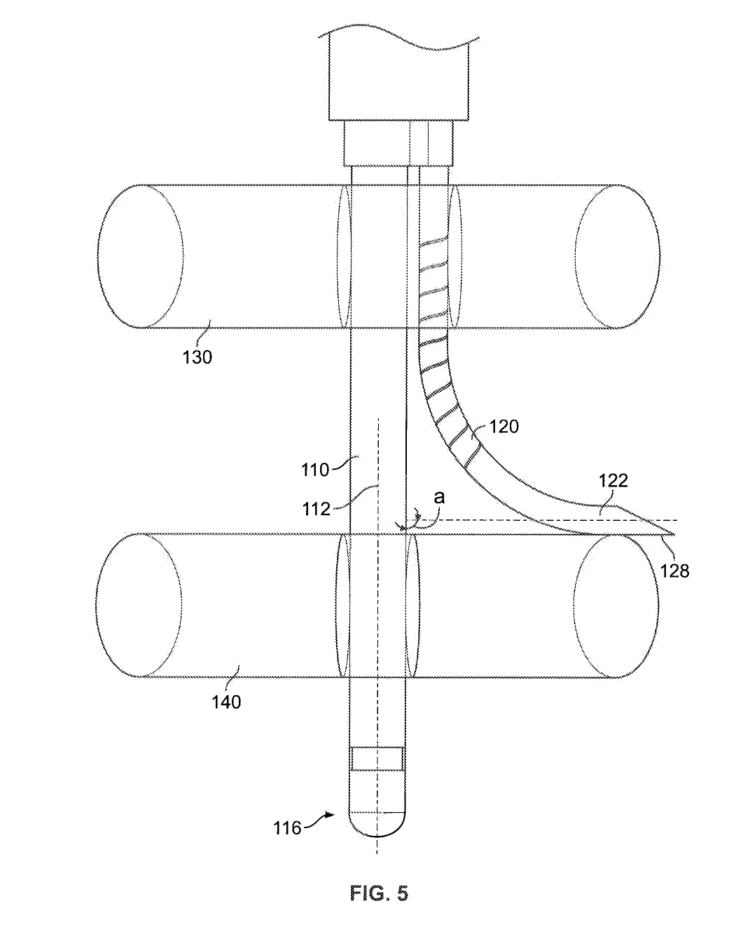

An embodiment of a catheter 100 presented herein is shown in FIGS. 2-7. In the embodiment shown, catheter 100 is a balloon biopsy device designed to biopsy tracheorbronchial, carinal and bronchopulmonary lymph nodes from the area around the trachea and primary bronchi. With reference to FIGS. 2-7, catheter 100 includes an outer sheath 102 serving as a delivery catheter that houses an inner catheter shaft 110 and a biopsy needle 120. As shown in FIG. 2, outer sheath 102 has a proximal end 104 and a distal end 106. As shown in FIG. 3, retraction of distal end 106 of outer sheath exposes inner catheter shaft 110 and biopsy needle 120 disposed side by side. Catheter shaft 110 has a proximal end 114 and a distal end 116, and biopsy needle has a proximal end 126 and a distal end 122 having a pointed tip 128 for puncturing targeted tissue to take a core sample during a biopsy procedure. In a retracted configuration of biopsy needle 120 in the embodiment of FIG. 3, inner catheter shaft 110 and biopsy needle 120 are disposed along side each other with their respective longitudinal axes in parallel. In an extended configuration of biopsy needle 120 as shown in FIG. 5, distal end portion 122 of needle 120 is bent to extend laterally from longitudinal axis 112 of catheter shaft 110 at a non-zero angle α relative to longitudinal axis 112, with respect to distal end 116 of shaft 110.

Outer sheath 102 and/or catheter shaft 110 can be provided with one or more location markers as known in the art to assist the physician in identifying the longitudinal position and/or rotational orientation of catheter 100 in a body lumen (e.g., trachea or bronchus) so that the physician can locate needle 120 proximate the targeted tissue in the body lumen and rotate catheter 100 to radially position needle 120 to face the targeted tissue, and to contact the targeted tissue when the needle is extended for biopsy. By way of example, as shown in FIG. 3, distal end 116 of catheter shaft 110 can be provided with one or more marker bands 118 configured to be seen with such imaging technologies as fluoroscopy and ultrasound.

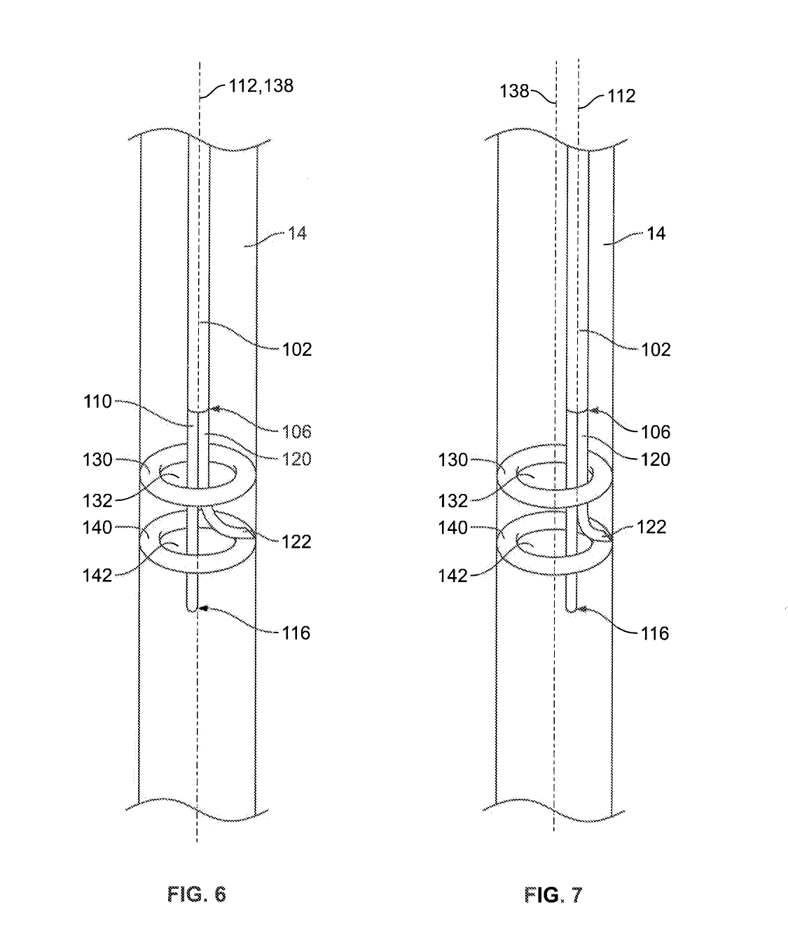

Catheter shaft 110 can be held securely in place the body lumen (e.g., trachea or bronchus) by means of one or more expandable support members. In the embodiment shown, catheter 100 includes first and second inflatable balloons 130 and 140 serving as expandable support members for securing catheter shaft 110 in the body lumen at the targeted area for biopsy. Balloons 130 and 140 are mounted on an outer surface of catheter shaft 110. Balloons 130 and 140 are donut-shaped so as to have an outer periphery with an central opening and an axial center (see openings 132 and 142 of balloons 130 and 140 and axial center 138 shown in FIGS. 6 and 7). Catheter shaft 110 and needle 120 extend through opening 132 of balloon 130 such that balloon 130 envelopes both catheter shaft 110 and needle 120 at their respective proximal ends 114 and 126. Catheter shaft 110 extends through opening 142 of balloon 140 such that balloon 140 envelopes only catheter shaft 110 at its distal end 116. Distal end 122 of needle 120 is adjacent to balloon 140 but remains outside the outer periphery of balloon 140. When balloons 130 and 140 are inflated to contact the wall of a body lumen (e.g., trachea or bronchus) and stabilize catheter shaft 110, respective openings 132 and 142 at the axial center 138 of balloons 130 and 140 permit the continued passage of bodily fluid or gas through the body lumen (e.g., inhaled or exhaled air through the trachea or bronchus) with minimal obstruction by the presence of inflated balloons 130 and 140, thereby reducing or eliminating the likelihood that the catheterization will detrimentally affect the patient's natural bodily functions.

In some embodiments, balloons 130 and 140 are made of a compliant material which allows the balloons to conform to the surface geometry of the body lumen, whereby the balloons can be expanded in the body lumen despite any irregularity in the dimensions of the body lumen relative to the donut-shape of the balloons, and avoid distorting or expanding the diameter of the body lumen. Thus, balloons 130 and 140 can provide sufficient friction with the body lumen walls to stabilize catheter shaft 110 therein without pressing against the lumen walls such force so as to cause distortion or requiring the body lumen to conform to the shape of the inflated balloon. Any compliant materials suitable for forming an inflatable balloon and biocompatible with a patient's body lumen may be used for balloons 130 and 140, such as soft polymeric materials including urethane, silicone, and combinations thereof, for example.

Although not shown, fluid supply lines are housed by catheter shaft 110 and join to balloons 130 and 140 for supplying fluid for balloon inflation as known in the art. The fluid supply lines can supply balloons 130 and 140 with biocompatible liquids (e.g., water or saline) or gas (e.g., air) for inflation, as known in the art. For bronchoscope applications of catheter 100, liquid inflation of balloons 130 and 140 can be hazardous to the patient's ability to breath if the balloon ruptures and releases the liquid in the airways, thus it should be apparent that the preferred fluid for inflating the balloons will vary depending on the catheterization procedures for which catheter 100 is used.

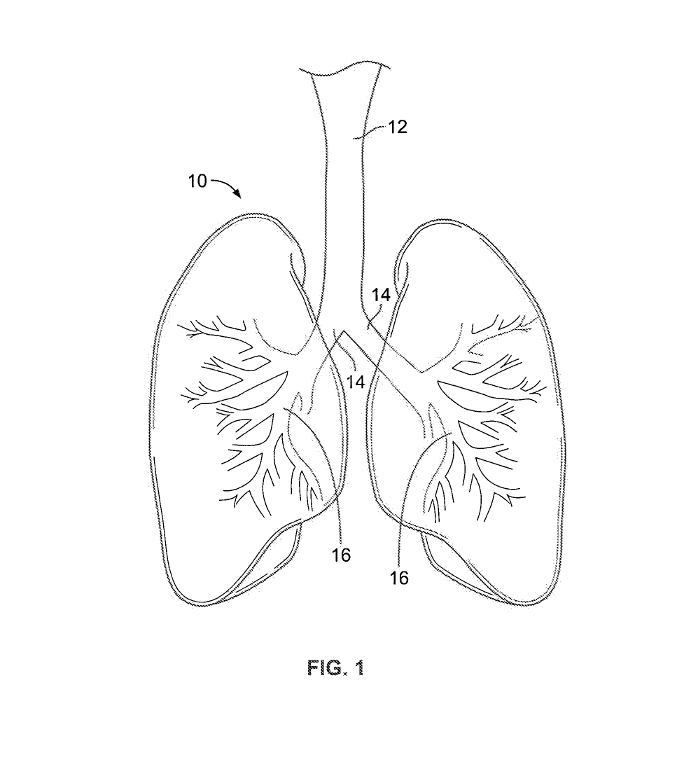

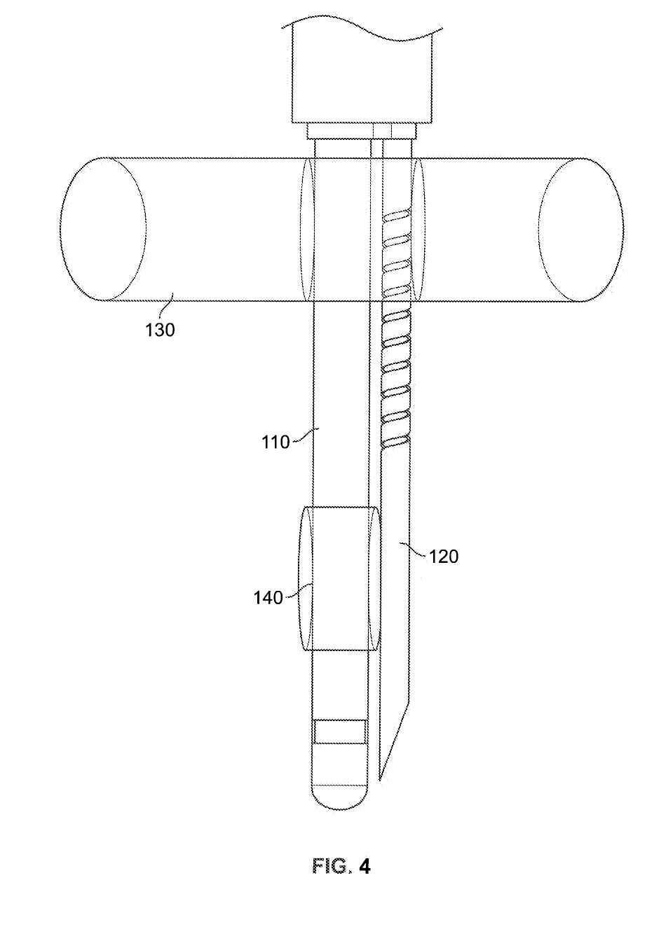

In some embodiments, balloons 130 and 140 can be independently inflatable, such as by using separate fluid supply lines to inflate respective balloons 130 and 140 to a selected diameter which can be same or different from each other. For example, as shown in FIG. 4, balloon 130 can be inflated first, thereby securing catheter shaft 110 in the desired position in the body lumen. Thereafter, balloon 140 can be inflated, as shown in FIG. 5, which can serve to further secure catheter shaft 110 in the body lumen. Further, in some embodiments, balloons 130 and 140 can be inflatable to different maximum diameters. For example, catheter 100 can be a flexible bronchoscope, and it may be desired to stabilize distal end 116 of inner catheter shaft 110 at the branch connection between one of the primary bronchi 14 and trachea 12 (see FIG. 1). In such an instance, distal end 116 of inner catheter shaft 110 can be located in the trachea and bronchus such that balloon 130 is positioned in trachea 12 and balloon 140 is positioned in bronchus 14. Since trachea 12 has a different luminal diameter (e.g., about 18 mm or generally in the range of from about 16 mm to about 19 mm for an adult trachea) than the luminal diameters of the primary bronchi (e.g., about 12 mm or generally in the range of from about 10 mm to about 15 mm for an adult primary bronchus), balloon 130 can be independently inflated to a larger diameter (e.g., about 18 mm) than the inflated diameter (e.g., about 15 mm) of balloon 140.

As shown in the embodiment of FIG. 5, when balloon 140 is inflated, balloon 140 contacts distal end 122 of needle 120, and bends distal end 122 of needle 120 laterally away from catheter shaft 110, deflecting distal end 122 to a non-zero angle α relative to longitudinal axis 112 of catheter shaft 110. Thus, the extent of inflation of balloon 140 can affect the amount distal end 122 of needle 120 is deflected by balloon 140. In some embodiments, the extent of inflation of balloon 140 can be varied, whereby the amount of deflection of distal end 122 of needle 120 by force of balloon 140 can also be varied. The inflation of balloon 140 can therefore be used to control and set the orientation of distal end 122 of needle 120 to the non-zero angle α which is relative to longitudinal axis 112 of catheter shaft 110, with respect to distal end 116 of shaft 110. In some embodiments, needle 120 is etched with laser cuts 124 at its proximal end 126 for making needle 120 easier to bend, thereby reducing the amount of force required to deflect needle to non-zero angle α. Likewise, non-zero angle α of distal end 122 of needle 120 can correspond with the angle at which the deflected distal end 122 is oriented relative the body lumen wall, since catheter shaft 110 is stabilized within the body lumen by at least balloon 130.

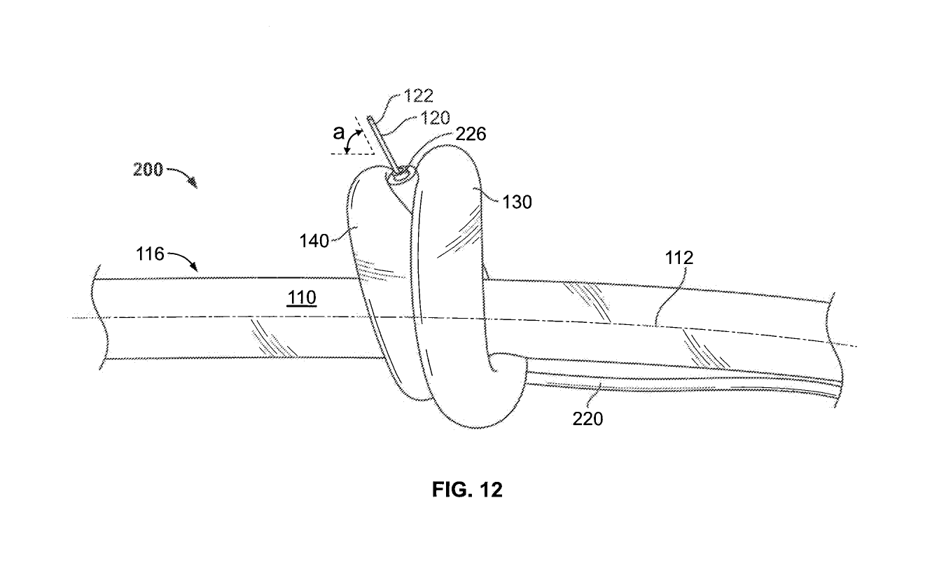

In the embodiment of FIG. 5, angle α is about 90°. In other embodiments, angle α can be about 60° (see, e.g., the embodiment of FIG. 12), in still other embodiments, angle α can be about 30°, about 35°, about 40°, about 45°, about 50°, about 55°, about 65°, about 70°, about 75°, about 85°, about 95°, about 100°, about 105°, about 110°, about 115°, about 120°, about 125°, about 130°, about 135°, about 140°, about 145°, and about 150° relative to longitudinal axis 112, with respect to distal end 116 of shaft 110. Thus, in some embodiments, inflation of balloon 140 can be inflated to various extents such that the amount of the inflation can be selected to control deflecting distal end 122 to a non-zero angle α within the range of from about 30° to about 150° relative to longitudinal axis 112. In still other embodiments, the amount of the inflation of balloon 140 can be selected to control deflecting distal end 122 to a non-zero angle α within the range of from about 30° to about 140°, from about 30° to about 130°, from about 30° to about 120°, from about 30° to about 110°, from about 30° to about 100°, from about 30° to about 90°, from about 40° to about 90°, from about 50° to about 90°, and from about 50° to about 70°.

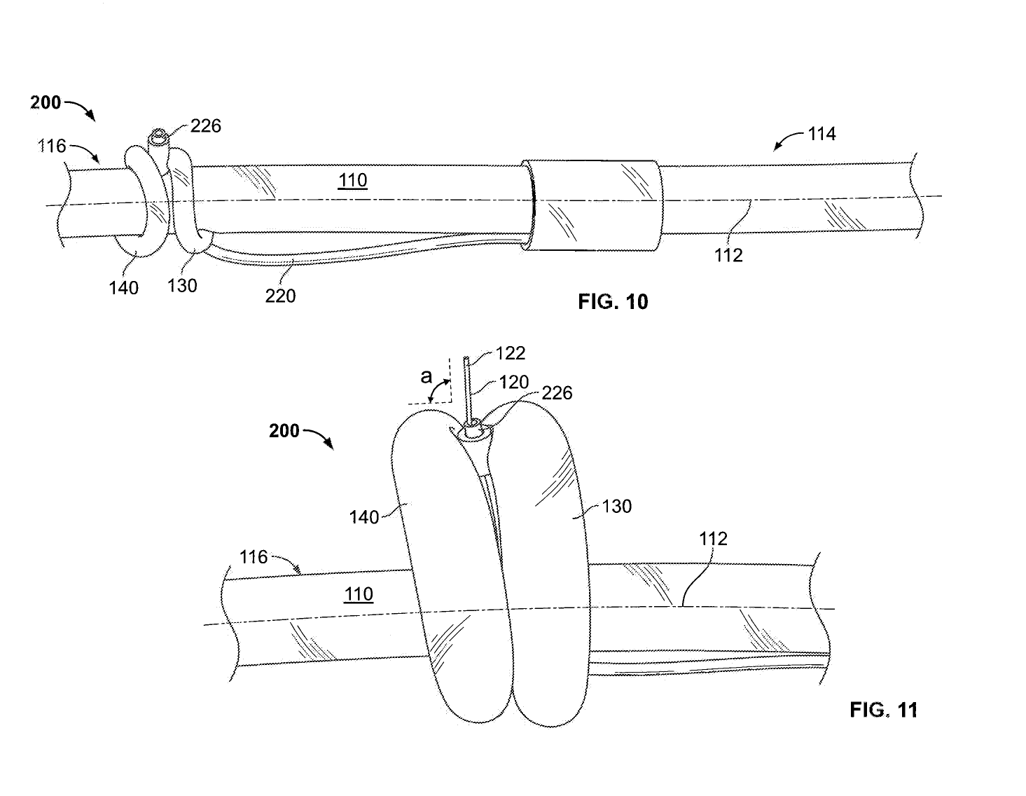

Thus, when catheter 100 extends in a body lumen, shaft 110 can be stabilized at a target location by at least balloon 130, and in some embodiments, can also be stabilized by balloon 140, provided balloon 140 is inflated an amount that not only achieves the selected deflection angle α, but also to coapt with the lumen wall to stabilize catheter shaft 110. Moreover, balloons 130 and 140 can be mounted on shaft 110 such that their axial center 138 is either concentric or eccentric with longitudinal axis 112 of catheter shaft 110. FIG. 6 illustrates an embodiment of catheter 100 stabilized in a main bronchus 14 with needle 120 deflected to engage the bronchial wall in which axial center 138 of inflated balloons 130 and 140 is concentric with longitudinal axis 112 of catheter shaft 110. In some cases, it maybe desirable to align catheter 100 eccentric to the centerline of the body lumen. FIG. 7 illustrates an embodiment of catheter 100 stabilized in a main bronchus 14 in which axial center 138 of inflated balloons 130 and 140 is eccentric with longitudinal axis 112 of catheter shaft 110. Thus, balloons 130 and 140 can be configured to position shaft 110 in the trachea or bronchus off center from centerline of the lumen of the trachea or bronchi, thereby allowing the physician to orient needle 120 closer to the side wall of the trachea or bronchus which can minimize entry point error when needle 120 is extended to take a core sample. The embodiment of FIGS. 10-14, described in detail below, also illustrates such an eccentric arrangement.

As shown in the embodiments of FIGS. 6 and 7, when balloons 130 and 140 are inflated to contact the bronchial wall of bronchus 14, respective openings 132 and 142 at the axial center 138 of balloons 130 and 150 permit the continued passage of air through the bronchus 14 lumen with minimal obstruction by the presence of inflated balloons 130 and 140. Thus, stabilization of catheter shaft 110 is achieved without restricting the airway in a manner that prohibits the proper breathing function of the patient. In some embodiments, the maximum cross-sectional area of the bronchi or trachea that can be obstructed by balloon 130 or 140 without inhibiting the proper breathing function of the patient will depend upon the condition of the patient and pulmonary function, and the amount of time for the procedure. For example, a procedure for an extended period of time would likely require the proper breathing function of the patient to be maintained during the procedure, whereas a very short procedure may allow the bronchi or trachea to be fully occluded during the procedure without adverse effects to the patient.

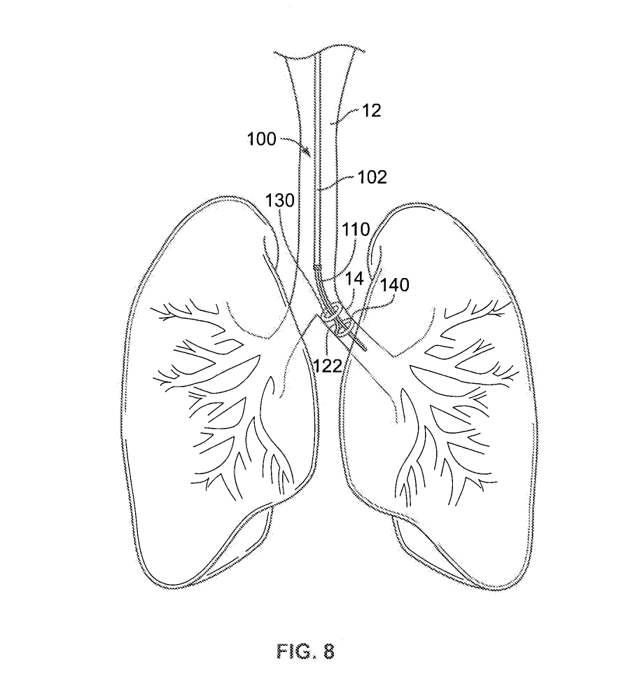

In operation, catheter 100 can be positioned in a body lumen and oriented using location markers so that needle 122 faces the targeted tissue area, and then balloons 130 and 140 can be inflated sequentially (or in some embodiments, simultaneously) to secure catheter 100 in the body lumen, and deflect needle 122 to non-zero angle α for contacting the targeted tissue area and taking a biopsy sample. For example, in the instance that catheter 100 is a bronchi biopsy device used to take a tissue sample of the bronchial lymph nodes, it is typically preferred that biopsy needle 120 is positioned 90° to the bronchial wall for penetration into the wall. For example, FIG. 8 illustrates catheter 100 located in a left main bronchus 14 with balloons 130 and 140 and needle 120 deflected to engage the bronchial wall at an angle α of about 90°. An exemplary procedure for using catheter 100 for bronchi biopsy can include inserting delivery catheter 102 into the patient's body through the nose or mouth, between the vocal cords and into the tracheobronchial tree. Distal end 106 can be disposed proximate the targeted lymph node tissue. Location markers on delivery catheter 102 and an imaging technique (e.g., fluoroscopy) can be used to visualize the location of distal end 106 of delivery catheter 102 to obtain accurate positioning. Distal end 106 of delivery catheter 102 can then be retracted to expose inner catheter shaft 110. Catheter 100 can be rotated to radially orient needle 120 to face the targeted tissue. Location markers on catheter shaft 110 can be used to visualize the rotational orientation of catheter shaft 110 relative to the bronchial wall so that needle 120 can be accurately oriented radially. Once longitudinal and rotational positioning is achieved, balloon 130 can be inflated to secure catheter shaft 110 in position. Balloon 140 can also be inflated a determined amount so as to simultaneously deflect needle 120 to angle α of 90°. Needle 122 can be slidably disposed on shaft 110, whereby needle can be slid distally by an amount as needed to cause the deflected distal end 122 to extend in the lateral direction away from the outer side of shaft 110 and engage the bronchial wall at 90° for taking a core sample of the targeted tissue.

It should be apparent that the catheters described herein can be modified to replace biopsy needle 120 or a cannula/needle system (e.g., cannula 220 with needle 120 such as described below with reference to FIGS. 10-15) with any other medical tool known in the art that is used for contacting a targeted location during a diagnostic or therapeutic catheterization procedure, and such modification would not depart from the scope of the present invention. For example, in some embodiments, the catheters described herein can be equipped with a needle configured to deliver a medicinal agent to a targeted tissue, which needle can be in place of or in addition to biopsy needle 120 or 220. In some embodiments, the catheters described herein can be equipped with a radiofrequency (RF) ablation needle provided with electrodes for RF ablation of targeted tissue (e.g., a malignant liver tumor) in place of or in addition to biopsy needle 120. Thus, the catheters described herein can be used for therapeutic treatment of a targeted tissue. In some embodiments in which the catheter includes an RF ablation needle, the RF ablation needle may include an inner channel through which a saline solution (i.e., a NaCl solution) is supplied to electrode(s) at the distal end of the RF ablation needle. In some embodiments, balloons 130 and/or 140 can be porous and be inflated with a saline solution, whereby the saline solution can percolate through the pores of the balloon(s) and contact the needle electrode and/or the targeted tissue prior to or during the ablation procedure. Any RF ablation needle known in the art can used with the catheters described herein. Exemplary RF ablation devices which can be employed with the catheters of the present invention, including devices configured to deliver RF energy and a saline solution, are described in U.S. Pat. Nos. 7,364,579; 6,949,098; 6,962,589; 6,327,505; 6,096,037; and 6,063,081, which are incorporated herein in their entirety by reference thereto.

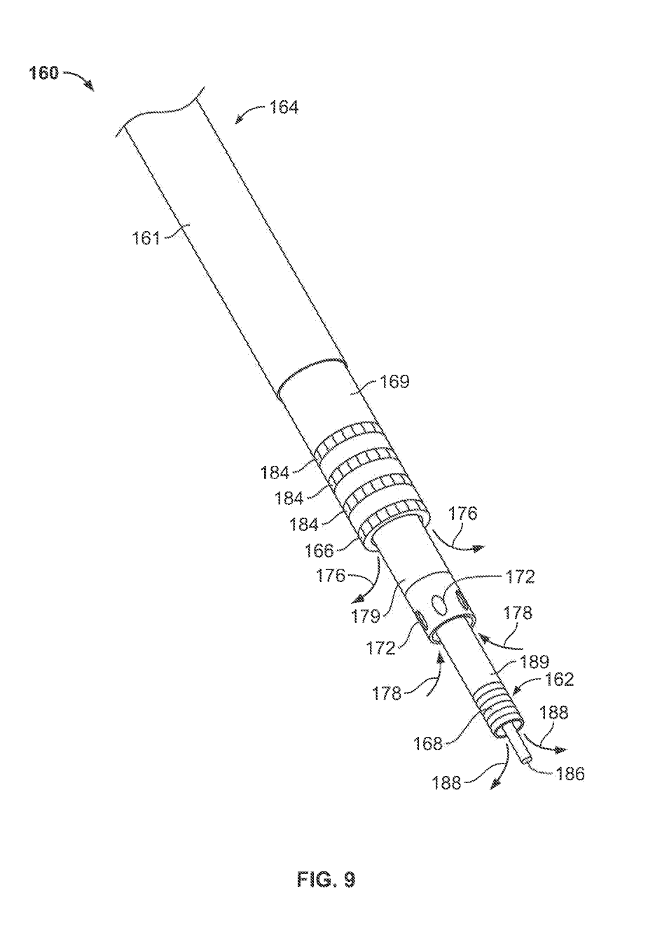

For example, FIG. 9 illustrates a distal end portion of an exemplary RF needle 160 which can be used in lieu of biopsy needle 120 of catheter 100, for delivering RF energy and a saline solution to a targeted tissue. RF needle 160 includes a proximal end portion 164 and a distal end portion 162, and three coaxially disposed tubes: an outer tube 169; a second, intermediate tube 179, and a third, inner tube 189. Outer tube 169 can be surrounded by an electrically insulative sheath 161 which terminates a short distance from a distal end of outer tube 169. Outer tube 169 provides a flow passage for an RF ablating fluid (e.g., a saline solution) from a fluid source (not shown) via proximal end portion 164 of needle 160 portion to the distal end of tube 169 where it exits as indicated by arrows 176. The distal end of outer tube 169 includes an electrode 166 and one or more thermocouples or other temperature sensors 184. Ablation needle 160 can include second, intermediate tube 179, which has a distal end provided with one or more vacuum apertures 172 through which a suction can be applied to surrounding tissue via a proximally located vacuum suction port (not shown). Suction will draw the surrounding tissue into contact with vacuum apertures 172 and the distal end of the second tube 179. In addition, with this same process, some RF ablating fluid may be removed via the applied suction as indicated by arrows 178. Ablation needle 160 can further include third, inner tube 189 having a distal end provided with an electrode 168, and a thermocouple 186 that extends distally from an interior passage of tube 189. The interior passage of tube 189 can provide a flow passage for the RF ablating fluid from the fluid source (not shown) to the distal end of tube 189, where the fluid exits as indicated by arrows 188. In using needle 160, electrodes 166 and 168 on respective tubes 169 and 189 can apply RF energy to targeted tissue, and the interior passages of tubes 169 and 189 can deliver the RF ablating fluid (e.g., a saline solution) into or onto the targeted tissue prior to or during an RF application. The infused saline solution can effectively improve tissue heating and increase the extent of necrosis during an RF application. Without wishing to be bound by an particular theory, it is believed that saline may enhance the efficiency of the RF ablation by enlarging the effective electrode surface area, improving the electric conductivity, allowing higher tolerance of sustained high generator output from tissue cooling and/or decreased impedance, and/or providing diffusion of heated saline into the tissue.

In some embodiments, needle 120 can be sheathed in a cannula and selectively extended from the cannula for contacting the targeted tissue via a body lumen. FIGS. 10-15 illustrate a catheter 200 according to an embodiment presented herein, in which needle 120 is slidably disposed in a lumen 222 of a flexible cannula 220 which has a distal end 226 that is sandwiched between inflatable balloons 130 and 140. In FIGS. 10-15, like reference numbers as previously described with regard to the embodiments of FIGS. 2-8 indicate identical or functionally similar elements, and therefore will not be described in detail again. Also, in FIGS. 10-14, outer delivery catheter 102 is not shown, but it should be understood that catheter 200 can include outer delivery catheter 102 if needed to introduce the other components of the biopsy device to the target location in the body.

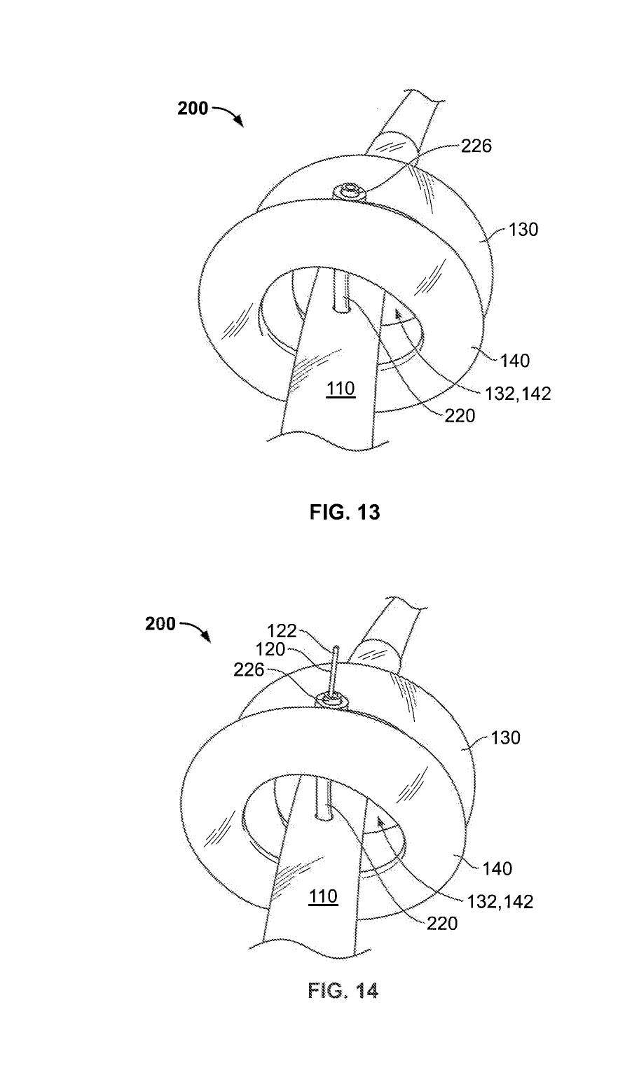

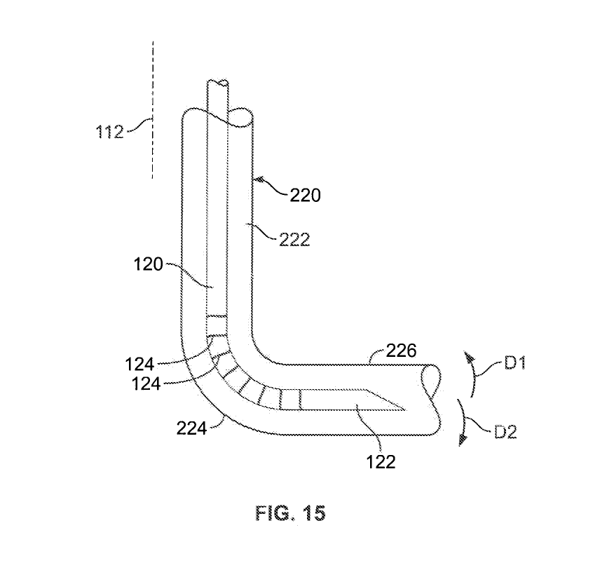

Referring to FIG. 10, flexible cannula 220 longitudinally extends along an outer side of catheter shaft 110, and has its distal end 226 sandwiched between deflated balloons 130 and 140 so as to laterally extend from the outer side of catheter shaft 110. As shown in FIG. 11, distal end 122 of needle 120 can be selectively extended from distal end 226 of cannula 220. As illustrated in FIGS. 13-15, needle 120 is slidably disposed in lumen 222 of cannula 220, so as to be selectively moved between a retracted position inside cannula (see FIGS. 13 and 15) and an extended position in which distal end 122 of needle 120 extends from cannula 220 (see FIG. 14). Moreover, in the embodiment shown of catheter 200, balloons 130 and 140 can be mounted on catheter shaft 110 so that their axial centers are eccentric with respect to the longitudinal axis 112 of catheter shaft 110 (best shown in the bottom view of FIGS. 13 and 14).

As shown in the schematic of FIG. 15, cannula 220 is bent at elbow portion 224 so that its distal end 226 extends laterally from longitudinal axis 112 of catheter shaft 110, and needle 120 takes the shape of cannula 220. Thus, deflection of distal end 226 at elbow portion 224 of flexible cannula 220 by an amount in either direction D1 and D2 will likewise deflect distal end 122 of needle 120 by the same amount, whereby the angle of the distal end 226 of cannula 220 relative to longitudinal axis 112 corresponds with the angle of distal end 122 of needle 120. Cannula 220 can be made to include elbow portion 224, or in some embodiments, flexible cannula 220 can be bent to include elbow portion when distal end 226 is sandwiched between balloons 130 and 140. In some embodiments, flexible cannula 220 can be disposed parallel with shaft 110 when balloons 130 and 140 are deflated (similar to the embodiment of FIG. 3), and inflation of balloon 140 actively bends cannula 220 at elbow portion 224 so that distal end 226 extends laterally from shaft 110 (similar to the embodiment of FIG. 5). In contrast with the balloon and needle configuration of embodiment shown in FIGS. 3-5, cannula 220 is sandwiched between balloons 140 and 130, with balloons 130 and 140 also pressing against distal end 226 of cannula 220.

Significantly, the amount of inflation of balloons 130 and 140 can be varied, and the amount of inflation of balloons 130 and 140 with respect to each other can control the non-zero angle α of distal end 226 of cannula 220 (and, likewise, distal end 122 of needle 120) relative to longitudinal axis 112 of catheter shaft 110. In addition, inflation of balloons 130 and 140 can stabilize cannula 220 disposed therebetween, which in turn can stabilize needle 120 and resist reaction forces arising from needle 120 contacting targeted tissue when taking a core sample.

In some embodiments, when balloons 130 and 140 have similar dimensions when inflated, distal end 226 of cannula (and, likewise, distal end 122 of needle 120) can be oriented at a non-zero angle α of about 90° relative to longitudinal axis 112, as shown in FIG. 12, for example. In some embodiments, balloon 140 can be inflated a lesser amount than that of balloon 130, whereby balloon 130 has a greater inflation diameter than balloon 140. In such embodiments, distal end 226 of cannula 220 sandwiched between balloons 130 and 140 can be deflected to an angle α less than 90° relative to longitudinal axis 112, with respect to distal end 116 of shaft 110, since larger balloon 130 presses distal end 226 distally against smaller balloon 140. For example, the relative inflation amounts of balloons 130 and 140 can deflect distal end 226 of cannula (and, likewise, distal end 122 of needle 120) to an angle α of about 60° with respect to distal end 116 of shaft 110, as shown in the embodiment of FIG. 12. Similarly, in some embodiments, balloon 140 can be inflated a greater amount than that of balloon 130, whereby balloon 140 has a greater inflation diameter than balloon 140. In such embodiments, distal end 226 of cannula 220 sandwiched between balloons 130 and 140 can be deflected to an angle α greater than 90° with respect to distal end 116 of shaft 110. Thus, in some embodiments, cannula 220 sandwiched between balloons 130 and 140 can be deflected to an angle α within the range of from about 30° to about 150° relative to longitudinal axis 112, with respect to distal end 116 of shaft 110. In still other embodiments, the amount of the inflation of balloons 130 and 140 can be selected to control distal end 226 of cannula 220 to a non-zero angle α within the range of from about 30° to about 140°, from about 30° to about 130°, from about 30° to about 120°, from about 30° to about 110°, from about 30° to about 100°, from about 30° to about 90°, from about 40° to about 90°, from about 50° to about 90°, and from about 50° to about 70°. In some embodiments, cannula 220 sandwiched between balloons 130 and 140 can be deflected to angle α of about 30°, about 35°, about 40°, about 45°, about 50°, about 55°, about 65°, about 65°, about 70°, about 75°, about 85°, about 95°, about 100°, about 105°, about 110°, about 115°, about 120°, about 125°, about 130°, about 135°, about 140°, about 145°, and about 150°. Thus, the orientation angle of the needle relative to the targeted tissue can be controlled for a given biopsy procedure via selective inflation amounts of balloons 130 and 140.

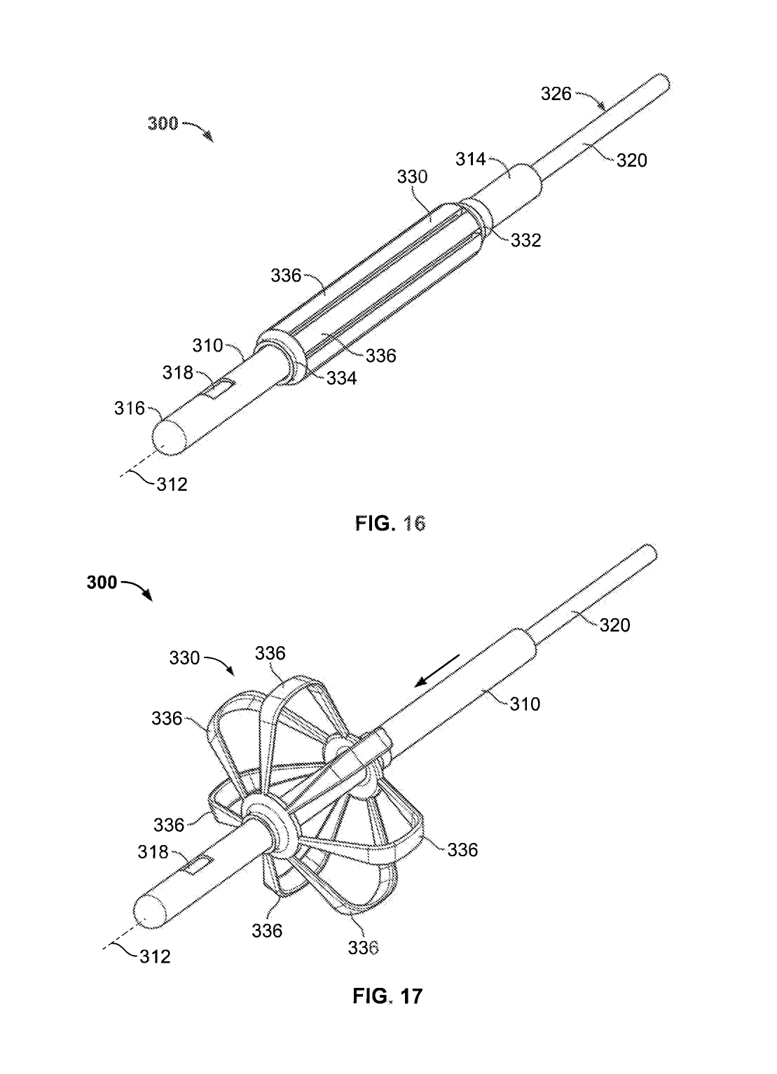

In any of the aforementioned embodiments, various structures other than inflatable balloons 130 and 140 can be used as expandable members for stabilizing catheters 100 or 200 and deflecting needle 120 to the selected non-zero angle α, thereby controlling the angle at which needle 120 contacts the body lumen wall during a diagnostic or therapeutic procedure. For example, in some embodiments, balloon 130 and/or 140 can be replaced with a non-inflatable mechanical structure, such as expandable members 330 or 430 formed from a shape-memory metal (e.g., nickel-titanium alloy) described further below with reference to the embodiments of FIGS. 16-24. In such an instance a strut 336 or 438 of the mechanical expandable members 330 or 430, respectively, can be configured to contact and deflect distal end 122 of needle 120 to the selected non-zero angle α.

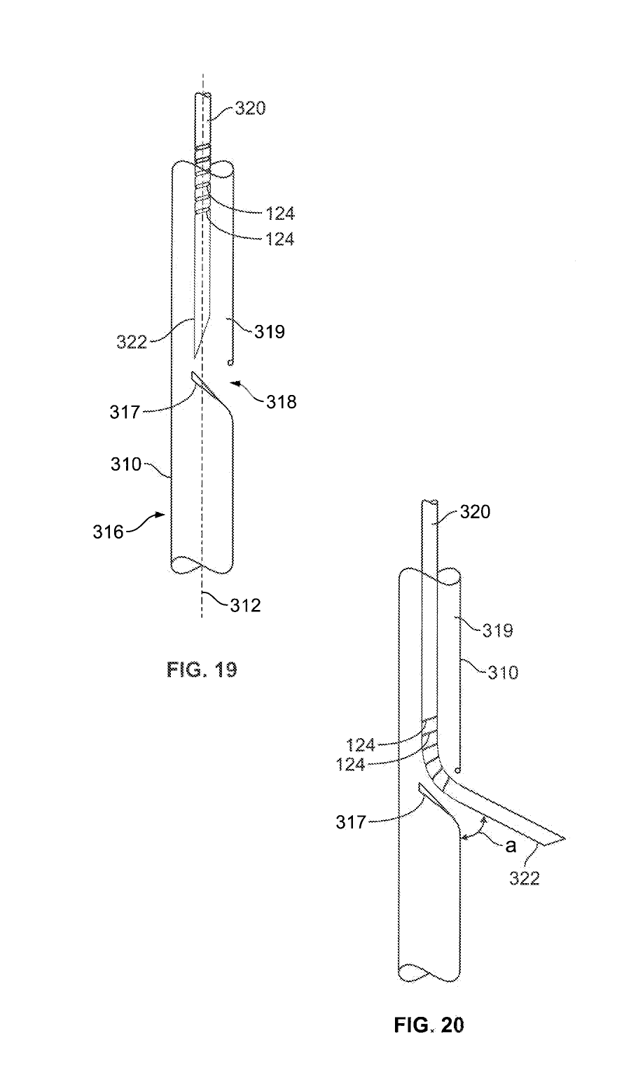

A bronchoscope 300 according to an embodiment of the present invention will now be described with reference to FIGS. 16-21. In some embodiments, bronchoscope 300 can be a flexible bronchoscope, and in other embodiments, bronchoscope 300 can be rigid bronchoscope. In the embodiment shown in FIGS. 16-21, bronchoscope 300 is a rigid bronchoscope, which includes a rigid bronchoscope shaft 310 having a proximal end 314 and an a distal end 316, a biopsy needle 320 having a proximal end 326 and a distal end 322, and a non-inflatable, mechanical expandable support member 330 preferably formed of a shape-memory material. Bronchoscope shaft 310 can also be provided with one or more marker bands 118 (see the embodiment of FIG. 3) or other location marker scheme known in the art. As shown in FIGS. 19 and 20, bronchoscope shaft 310 has a lumen 319 in which is a biopsy needle 320 is slidably disposed, and a needle port 318 is provided in shaft 310 at its distal end 316 through which distal end 322 of biopsy needle 320 can exit. Expandable support member 330 includes a distal skirt 334 fixedly mounted shaft bronchoscope 310, a proximal skirt 332 spaced from distal skirt 334 and selectively slidably mounted on shaft 310, and a plurality of discrete struts 336 extending from skirt 332 to skirt 334.

As shown in FIG. 17, when skirt 332 is slid distally along the longitudinal axis 312 of shaft 310 (as shown by the direction of the arrow), struts 336 extend radially outwardly from shaft 310 to assume a pre-formed shape of the shape-memory material. Any shape-memory material known in the art can be used for forming expandable support member 330, including, for example a nickel-titanium (NiTi) alloy such as NITINOL, and shape memory materials made of stainless steel or cobalt alloy, including for example NP35N and NP35NLT. Alternatively, in some embodiments, proximal skirt 332 can be fixedly mounted shaft bronchoscope 310 and distal skirt 334 can be selectively slidably mounted on shaft 310.

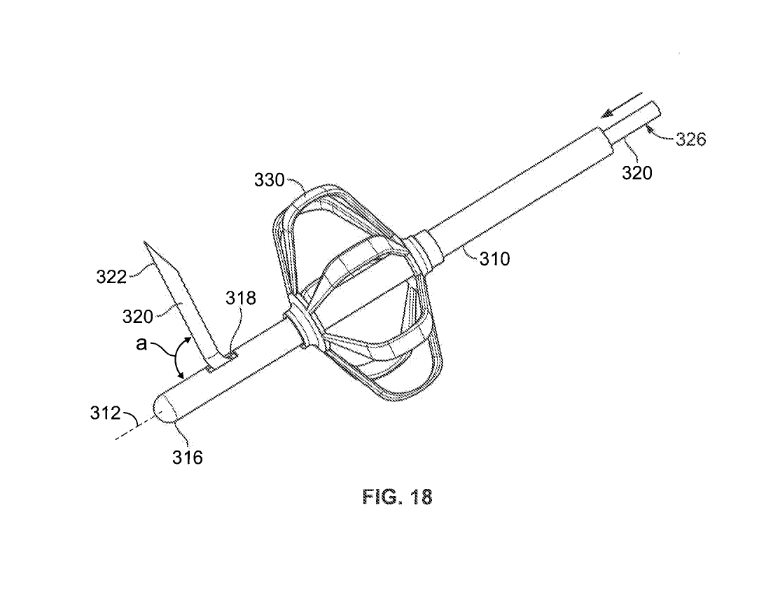

As shown in FIG. 18, when biopsy needle 320 is slid distally in lumen 319 along the longitudinal axis 312 of shaft 310 (as shown by the direction of the arrow), distal end 322 extends through port 318. Any biopsy needle known in the art can be used as biopsy needle 320. In some embodiments, biopsy needle 320 is a hollow coring needle, as known in the art. Preferably, biopsy needle 320 is one that can be bent at its distal end to assume a non-zero angle relative to longitudinal axis 312 and can be straightened without fracturing when retracted within lumen 319. In some embodiments, biopsy needle 320 can be formed of a shape-memory material, for example as a nickel-titanium alloy such as NITINOL, which is provided with a heat set bent distal end 322. Thus, when distal end 322 extends from lumen 319 via port 318, distal end 322 can assume the heat set bent shape so as to extend laterally from the longitudinal axis of shaft 310 at a non-zero angle α with respect to distal end 316 of shaft 310.

Alternatively or in addition to providing a pre-formed bend in distal end 322, bronchoscope shaft 310 may include a ramp 317 extending from a distal side of port 318 into lumen 319 (see FIGS. 19 and 20). The incline of ramp 317 can be configured to assist bending distal end 322 of needle 320 when exiting port 318 so that distal end 322 extends laterally from the longitudinal axis of shaft 310 at the non-zero angle α. Needle 320 can further include laser cuts 124 positioned at locations immediately adjacent distal end 322 to facilitate bending distal end 322 to the non-zero angle α. In some embodiments, ramp 317 and/or the shape-memory material of the needle 320 are configured such that distal end 322 of needle 320 extends from port 318 at a non-zero angle α of about 90° relative to longitudinal axis 312. In some embodiments, ramp 317 and/or needle can be configured so that the non-zero angle α is in the range of from about 30° to about 90°, and in other embodiments, ramp 317 and/or needle can be configured so that the non-zero angle α is in the range of from about 90° to about 150° with respect to distal end 316 of shaft 310. Depending on the stress and strain properties of the shape-memory material forming needle 320, a 90° or greater bend may not be obtainable without fracturing the needle when it is retracted within lumen 312. As should be apparent to one skilled in the art, the ability to bend the needle to a given non-zero angle α such that the needle withstands fracture when retracted within lumen 312 requires designing the needle within the limits of the material forming the needle. For example, for a needle formed of NITINOL, the radius of a permissible bend is typically constrained to the needle diameter by a ratio of 7:1, in which one unit increase in diameter increases the bend radius of bend seven-fold. However, laser cuts 124 can be used to improve this 7:1 ratio by reducing the permissible bend radius for a given needle diameter. Thus, for example, in some embodiments, needle 320 can have a heat set bend at its distal end 322 of no more than about 60°. In such embodiments, a non-zero angle α of greater than about 60° (e.g., 75°) can be achieved by configuring laser cuts 124 and the incline of ramp 317 so that distal end 322 when exiting port 318 is pushed by ramp 317 to further flex at laser cuts 124 to achieve a selected non-zero angle α greater than about 60°.

In some embodiments, bronchoscope 300 can include one or more additional expandable support members structured similar to expandable support member 330. For example, FIG. 21 illustrates bronchoscope 300 including a second expandable support member 340 configured the same as first expandable member 330 but located distally of expandable member 330 so that needle 320 is interposed between first expandable member 330 and second expandable member 340. Second expandable member 340, if provided, can serve to further secure bronchoscope shaft 310 in position and resist movement of distal end 316 of shaft 310 from the reaction forces from needle 320 contacting targeted tissue when taking a core sample. For example, as shown in FIG. 21, bronchoscope 300 can be located in the trachea 12 with each of expandable members 330 and 340 in an expanded position. The distal end 322 of needle 320 can then be extended from shaft 310 to contact the portion of the side wall of trachea 12 which located between expandable members 330 and 340.

In operation, bronchoscope 300 can be used to take a core sample from the trachea or primary bronchi. An exemplary procedure for using rigid bronchoscope 300 for taking a biopsy of the lymph nodes located around the trachea and primary bronchi can include inserting bronchoscope shaft 310 (with expandable support member 330 (and member 340, if provided) being in a retracted position as shown in FIG. 16) into the patient's body through the mouth, between the vocal cords and into the tracheobronchial tree. Distal end 316 can be disposed proximate the targeted tissue. Location markers on bronchoscope shaft 310 and an imaging technique (e.g., fluoroscopy) can be used to visualize the location of distal end 316 of bronchoscope shaft 310 to obtain accurate positioning. Bronchoscope shaft 310 can be rotated to orient port 318 to face the targeted tissue. Location markers on bronchoscope shaft 310 can be used to visualize the rotational orientation of bronchoscope shaft 310 relative to the targeted tissue of trachea 12 so that the radial orientation of needle 320 within the tracheal lumen can be confirmed.

Once longitudinal and rotational positioning is achieved, expandable member 330 (and expandable member 340, if provided) can be expanded to secure bronchoscope shaft 310 in position. For example, proximal skirt 332 of expandable member 330 can be linked to a tool disposed outside of the patient that the physician can manipulate to slide skirt 332 distally along the longitudinal axis 312 of shaft 310 so that struts 336 extend radially outwardly from shaft 310 to assume an expanded configuration corresponding to the pre-formed shape of the shape-memory material, as shown in FIG. 17. Needle 320 can also be linked to a tool disposed outside of the patient that the physician can manipulate to slide needle 320 distally in lumen 319 along the longitudinal axis 312 of shaft 310. Sliding of needle 320 forces distal end 322 to extend through port 318, whereby distal end 322 can contact the targeted tissue and take a core sample. Any heat set angle in distal end 322, along with the assist from ramp 317 and laser cuts 124, causes the extended distal end 322 of needle 320 to deflect to a non-zero angle α relative to longitudinal axis 312 of shaft 310 (see, e.g., FIGS. 18 and 20), whereby the orientation angle of the needle upon delivery to the targeted tissue is known.

Moreover, when expandable member 330 is manipulated to the expanded configuration, struts 336 splay apart so as to include open areas between the struts through which air can flow. A patient's breathing is therefore not detrimentally restricted during a bronchoscopy biopsy procedure using bronchoscope 300. Expandable member 330 permits secure and accurate placement of bronchoscope shaft 310 eccentrically within the lumen of the trachea or bronchi, but still allows the continued passage of air past bronchoscope 300.

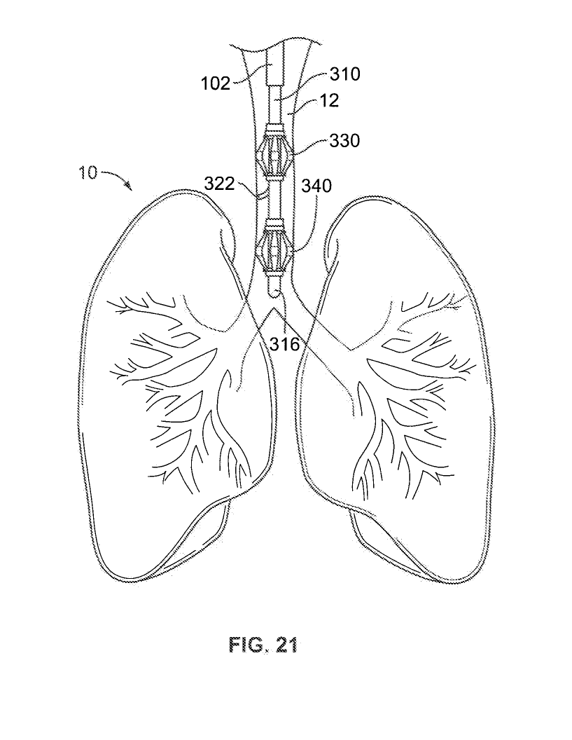

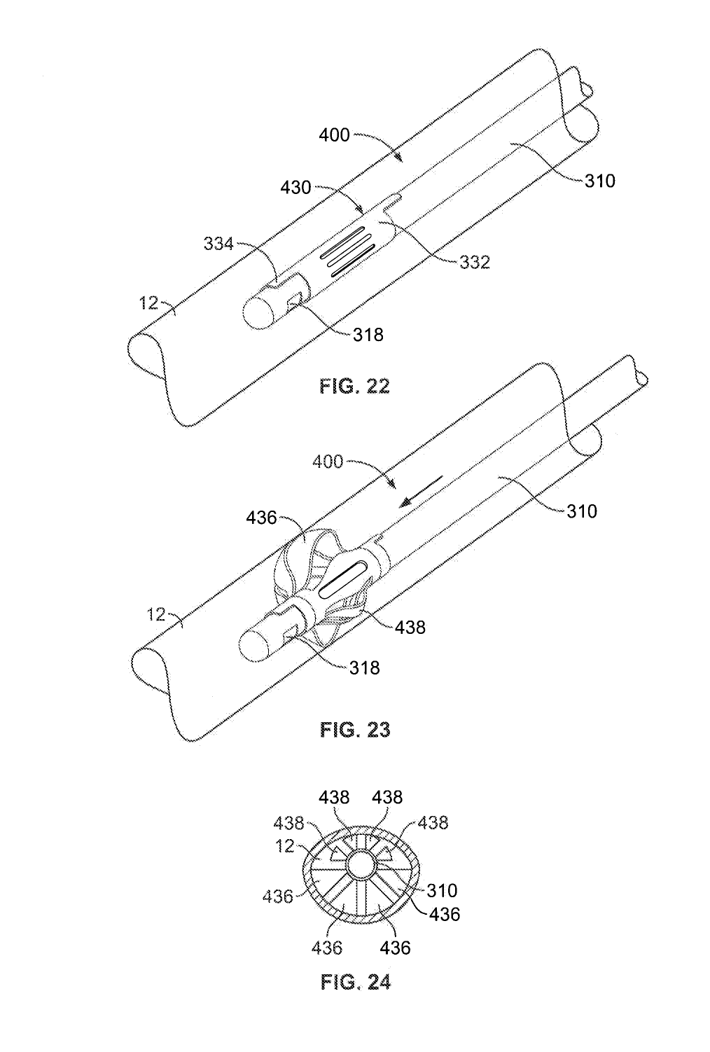

In some cases, it maybe desirable to align the bronchoscope eccentric to the lumen of the trachea or bronchi. FIGS. 22-24 illustrate a bronchoscope 400 according to an embodiment of the present invention, which is similar to bronchoscope 300 but which includes an eccentric expandable member 430 instead of expandable member 330. Eccentric expandable member 430 positions bronchoscope shaft 310 in the trachea or bronchus off center from centerline of the lumen of the trachea or bronchi, thereby allowing the physician to orient needle port 318 close to the side wall of the trachea or bronchus. The ability to locate shaft 310 eccentric to the tracheal/bronchi lumen allows port 318 to be situated close to the lumen wall, thereby minimizing entry point error when needle 320 is extended to take a core sample. Thus, this close alignment further assures accurate placement of needle 320 with respect to the targeted tissue. In FIGS. 22-24, like reference numbers as previously described with regard to the embodiments of FIGS. 16-21 indicate identical or functionally similar elements, and therefore will not be described in detail again. Also, in FIGS. 22-24, a biopsy needle is not shown, but it should be understood bronchoscope includes needle 320 as described above with references to the embodiments of FIGS. 16-21.

FIGS. 22-24 illustrate bronchoscope 400 disposed in trachea 12. Eccentric expandable member 430 is mounted on shaft 310 at proximate side of needle port 318. Similar to previously described expandable member 330, any shape-memory material known in the art can be used for forming expandable support member 430, including, for example a nickel-titanium (NiTi) alloy such as NITINOL. Eccentric expandable member 430 has distal skirt 334 fixedly mounted shaft bronchoscope 310 and proximal skirt 332 spaced from distal skirt 334 and selectively slidably mounted on shaft 310. Alternatively, in some embodiments, proximal skirt 332 can be fixedly mounted shaft bronchoscope 310 and distal skirt 334 can be selectively slidably mounted on shaft 310.

In contrast with expandable member 330, eccentric expandable member 430 is provided with a plurality of first struts 436 and a plurality of second struts 438 which extend from skirt 332 to skirt 334. As shown in FIG. 23, when skirt 332 is slid distally along the longitudinal axis 312 of shaft 310 (as shown by the direction of the arrow), struts 436 and 438 extend radially outwardly from shaft 310 to assume a pre-formed shape of the shape-memory material. Struts 436 are longer in length than struts 438, such that when expandable member 430 is slid to the expanded position shown in FIG. 23, longer struts 436 contact the side wall of trachea 12 so as to shift shaft 310 off the longitudinal centerline of trachea 12, thereby positioning shaft 310 eccentrically within trachea 12. Struts 436 are disposed around a portion of the radial perimeter of shaft 310 on a radially opposite side from where needle port 318 is disposed, and struts 438 are disposed around the remaining portion of the radial perimeter of shaft 310 so as to be on the same side as port 318, as best shown in FIGS. 23 and 24. Eccentrically positioned shaft 310 is secured in a spaced relationship from the side wall by the expanded struts 438 and struts 436 holding shaft 310 in place. However, needle port 319 is on the side of shaft 310 that is closer to the side wall of trachea 12 since shorter struts 438 are disposed on the same side as needle port 318.

Moreover, similar to expandable member 330, struts 436 and 438 splay apart in the expanded configuration so as to include open areas between the struts through which air can flow. This assures that a patient's breathing is not detrimentally restricted when bronchoscope 400 is deployed in a patient's airway during a bronchoscopy biopsy procedure. Thus, expandable member 430 permits secure and accurate placement of bronchoscope shaft 310 eccentrically within the trachea or bronchus, but still allows adequate amounts of air to flow through the trachea or bronchus despite expandable member 430 being expanded in the airway. The operation of bronchoscope 400 is the same as bronchoscope 300 described with reference FIGS. 16-21 and therefore will not be described in detail again.

The foregoing description of the specific embodiments of the devices and methods described with reference to the Figures will so fully reveal the general nature of the invention that others can, by applying knowledge within the skill of the art, readily modify and/or adapt for various applications such specific embodiments, without undue experimentation, without departing from the general concept of the present invention. For example, in some embodiments, a second eccentric expandable member similar to member 430 can be provided on shaft 410 of bronchoscope 400, in a similar manner as second expandable member 340 is provided on shaft 310 of bronchoscope 300 in the embodiment illustrated in FIG. 21. In addition, as those of skill in the art will readily understand, expandable members 330, 340 or 430 can be replaced with inflatable balloon structures for stabilizing bronchoscopes 300 and 400. For example, in some embodiments, expandable members 330 and/or 340 can be replaced with balloons 130 and/or 140 described above with reference to the embodiments of FIGS. 2-8 and 10-14. In some embodiments, eccentric expandable member 430 of the embodiment of FIGS. 22-24 can be replaced with one or more eccentrically oriented inflatable balloon such as eccentrically oriented balloon 130 or 140 described with reference to in FIG. 7.

In some embodiments, catheters 100 and 200, and bronchoscopes 300 and 400, can optionally be provided with mechanisms for imaging the interior of a body lumen, such as a vision system (including, e.g., a fiberoptic system) and/or a fluoroscopic imaging system.

Thus, various structures other than those disclosed herein may be used for stabilizing the catheters of FIGS. 2-24 within a body lumen to facilitate advancement of a needle therefrom, and in some embodiments, to control the angle at which the extended needle contacts the targeted tissue, with or without the assistance of imaging techniques.

Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance. The breadth and scope of the present invention should not be limited by any of the above-described exemplary embodiments, but should be defined only in accordance with the following claims and their equivalents.

What is claimed is:

1. A catheter for insertion into a body lumen, comprising:
an elongated catheter shaft having a proximal end portion, a distal end portion, a radial perimeter, and a longitudinal axis;
an expandable member coupled to an outer surface of the distal end portion of the catheter shaft, the expandable member extending around the radial perimeter of the catheter shaft and being selectively radially expandable from the radial perimeter of the catheter shaft for making contact with a body lumen wall; and
a needle having a distal end adjacent the expandable member and disposed on an outer side of the distal end portion of the catheter shaft, the distal end of the needle being configured to extend laterally from the outer side of the catheter shaft at a non-zero angle relative to the longitudinal axis of the catheter shaft, wherein the expandable member contacts the needle during the selective radial expansion of the expandable member such that the non-zero angle of the extended needle is controlled by the expansion of the expandable member.

2. The catheter of claim 1, wherein a radial center of the expandable member is eccentrically arranged with respect to the longitudinal axis of the catheter shaft so that, when the expandable member is expanded in a body lumen, the distal end portion of the catheter shaft is off-centered in the body lumen.

3. The catheter of claim 1, wherein the expandable member includes at least one opening for allowing bodily fluid or gas to pass through when the expandable member is expanded in a body lumen.

4. The catheter of claim 1, wherein the expandable member is an inflatable balloon, and wherein an amount of inflation of the balloon controls the non-zero angle of the extended needle, wherein, when the balloon is selectively inflated to a first amount, the balloon moves the needle to a first non-zero angle relative to the longitudinal axis of the catheter shaft, and wherein, when the balloon is selectively inflated to a second amount, the balloon moves the needle to a second non-zero angle which is relative to the longitudinal axis of the catheter shaft.

5. The catheter of claim 4, wherein the first amount of inflation is greater than the second amount of inflation, and wherein the first non-zero angle of the needle is larger than the second non-zero angle of the needle.

6. The catheter of claim 1, further comprising:
a second expandable member coupled to an outer surface of the distal end portion of the catheter shaft and spaced from the first expandable member, the second expandable member extending around the radial perimeter of the catheter shaft and being selectively radially expandable from the radial perimeter of the catheter shaft for making contact with a body lumen wall,
and wherein the extended needle is interposed between the first expandable member and the second expandable member.

7. The catheter of claim 6, wherein a radial center of the second expandable member is eccentrically arranged with respect to the longitudinal axis of the catheter shaft.

8. The catheter of claim 6, wherein the second expandable member includes at least one opening for allowing bodily fluid to passes through when the second expandable member is expanded in a body lumen.

9. The catheter of claim 6, wherein at least one of the first and second expandable members are inflatable balloons made of a compliant material.

10. The catheter of claim 9, wherein the compliant material includes silicone.

11. The catheter of claim 6, wherein the first and second expandable members are respective first and second inflatable balloons.

12. The catheter of claim 11, wherein an amount of inflation of the first and second balloons controls the non-zero angle of the extended needle, wherein the first balloon is configured to be selectively inflated to a first amount, and wherein the second balloon is configured to be selectively inflated to a second amount, wherein, when the first balloon is selectively inflated to the first amount and the second balloon is inflated to the second amount, the first and second balloons move the needle to a first non-zero angle relative to the longitudinal axis of the catheter shaft.

13. The catheter of claim 12, wherein the first amount of inflation of the first balloon is the same as the second amount of inflation of the second balloon, and wherein the first non-zero angle of the needle is about 90 degrees.

14. The catheter of claim 12, wherein the first amount of inflation of the first balloon is greater than the second amount of inflation of the second balloon, and wherein the first non-zero angle of the needle is about 60 degrees.

15. The catheter of claim 12, wherein the second balloon is configured to be selectively inflated to a third amount, wherein, when the second balloon is inflated to the third amount, the first and second balloons move the needle to a second non-zero angle relative to the longitudinal axis of the catheter shaft.

16. The catheter of claim 15, wherein the first amount of inflation of the first balloon is the same as the second amount of inflation of the second balloon, and wherein the second amount of inflation of the second balloon is greater than the third amount of inflation of the second balloon, and wherein the second non-zero angle of the needle is less than about 90 degrees.

17. The catheter of claim 16, wherein the second non-zero angle of the needle is about 60 degrees.

18. The catheter of claim 6, wherein at least one of the first and second expandable members is formed from a shape-memory material.

19. The catheter of claim 1, wherein the needle has a retracted position in which the distal end of the needle is parallel with the longitudinal axis of the catheter shaft.

20. The catheter of claim 1, wherein the needle is a biopsy needle.

21. The catheter of claim 1, wherein the needle is a radiofrequency (RF) ablation needle.

22. The catheter of claim 21, wherein the RF ablation needle is configured to deliver RF energy and a saline solution.

23. The catheter of claim 1, wherein the needle comprises a flexible cannula and an inner needle, the inner needle having a distal end slidably disposed in the cannula between a retracted position inside the cannula and an extended position extending outside the cannula.

24. The catheter of claim 23, wherein the inner needle is a biopsy needle.

25. The catheter of claim 23, wherein the inner needle is a radiofrequency ablation needle.

26. The catheter of claim 1, wherein the distal end portion of catheter shaft is provided with a location marker.

27. The catheter of claim 1, wherein the expandable member is a non-inflatable expandable member having a proximal skirt, a distal skirt longitudinally spaced from the proximal skirt, and a plurality of elongated struts extending from the proximal skirt to the distal skirt and extending around the radial perimeter of the catheter shaft, wherein one of the proximal and distal skirts is fixedly mounted on the catheter shaft, and wherein the other of the distal and proximal skirts is slidably mounted on the catheter shaft such that the expandable member has a retracted position and expanded position in which the plurality of elongated struts radially expand from the radial perimeter of the shaft for making contact with the body lumen wall.

28. A catheterization method for maneuvering a needle to contact a targeted tissue, comprising:
introducing a catheter into a body lumen, the catheter including:
an elongated catheter shaft having a proximal end portion, a distal end portion, a radial perimeter, and a longitudinal axis,
an expandable member coupled to an outer surface of the distal end portion of the catheter shaft, the expandable member extending around the radial perimeter of the catheter shaft and being selectively radially expandable from the radial perimeter of the catheter shaft for making contact with a body lumen wall, and
a needle connected to the catheter shaft and having a distal end proximate the distal end portion of catheter shaft, the distal end being laterally extendable from a side of the catheter shaft;
positioning the distal end portion of the catheter shaft so that the distal end of the needle faces opposite a target location on the body lumen wall; and
expanding the expandable member in the body lumen to contact the body lumen wall, wherein expandable member contacts the needle during expansion and the expandable member moves the needle to laterally extend to a non-zero angle relative to the longitudinal axis of the catheter shaft.

29. The method of claim 28, wherein the needle is slidable along the longitudinal axis of the catheter shaft, the method further comprising:
after expanding the expandable member, sliding the needle distally by an amount which forces the distal end of the needle against the target location on the body lumen wall.

30. The method of claim 28, wherein the non-zero angle is an angle in the range of from about 30 degrees to about 150 degrees relative to the longitudinal axis of the catheter shaft.

31. The method of claim 28, a radial center of the expandable member is eccentrically arranged with respect to the longitudinal axis of the catheter shaft, wherein expanding the expandable member causes the distal end portion of the catheter shaft to be off-center in the body lumen.

32. The method of claim 28, wherein the expandable member is an inflatable balloon, wherein expanding the expandable member comprises inflating the balloon with a fluid, wherein the fluid comprises one of air and a liquid.

33. The method of claim 32, wherein inflating the balloon comprises inflating the balloon to contact the body lumen wall so that that the inflated balloon conforms to a surface geometry of the body lumen without expanding a diameter of the body lumen.

34. The method of claim 32, wherein inflating the balloon comprises:
inflating the balloon by an first amount to move the needle to a first non-zero angle relative to the longitudinal axis of the catheter shaft; and
inflating the balloon by a second amount to move the needle to a second non-zero angle relative to the longitudinal axis of the catheter shaft,
wherein the first amount of inflation is greater than the second amount of inflation, and wherein the first non-zero angle of the needle is larger than the second non-zero angle of the needle.

35. The method of claim 28, wherein the expandable member includes a first expandable member and a second expandable member, the second expandable member being spaced from the first expandable member, wherein the distal end of the needle is interposed between the first expandable member and the second expandable member, wherein the first and second expandable members are respective first and second inflatable balloons, wherein expanding the expandable member comprises:
inflating the first balloon to a first amount;
inflating the second balloon to a second amount,
wherein the first and second inflated balloons move the needle to the non-zero angle relative to the longitudinal axis of the catheter shaft.

36. The method of claim 35, wherein the first amount of inflation of the first balloon is the same as the second amount of inflation of the second balloon, and wherein the non-zero angle of the needle is about 90 degrees.

37. The method of claim 35, wherein the first amount of inflation of the first balloon is greater than the second amount of inflation of the second balloon, and wherein the non-zero angle of the needle is about 60 degrees.

38. A bronchoscope comprising:
an elongated tubular shaft having proximal end portion, a distal end portion, a radial perimeter, a longitudinal axis, a lumen, a needle port disposed at the distal end portion of shaft, and a ramp, wherein the shaft defines the lumen which communicates with the needle port, and wherein the ramp extends from a distal side of the needle port into the lumen;
a needle having a distal end portion and slidably disposed in the lumen such that the needle has a retracted configuration and an extended configuration in which the distal end portion of the needle contacts the ramp and extends laterally through the needle port at a non-zero angle relative to the longitudinal axis of the shaft; and
a non-inflatable expandable member coupled to an outer surface of the distal end portion of the shaft, the expandable member having a proximal skirt, a distal skirt longitudinally spaced from the proximal skirt, and a plurality of elongated struts extending from the proximal skirt to the distal skirt and extending around the radial perimeter of the shaft,
wherein one of the proximal and distal skirts is fixedly mounted on the shaft, and wherein the other of the distal and proximal skirts is slidably mounted on the shaft such that the expandable member has a retracted position and expanded position in which the plurality of elongated struts radially expand from the radial perimeter of the shaft for making contact with a lumen wall of an airway of a tracheobronchial tree, and
wherein the plurality of struts splay apart away from each other when the expandable member is moved from the retracted position to the expanded position, and wherein an opening is provided between each of the splayed apart struts for allowing air to pass through when the expandable member is in the expanded position in the airway.

39. The bronchoscope of claim 38, wherein the plurality of struts include first struts and second struts, wherein the first struts have a longer length than a length of the second struts, wherein the first struts are positioned on a radially opposite side from where the needle port is disposed on the shaft, and wherein the second struts are disposed around the remaining portion of the radial perimeter of shaft so as to be on the same side as the needle port, wherein, when the expandable member is expanded in the body lumen, the longitudinal axis of the shaft is eccentrically arranged with respect to a longitudinal axis of the body lumen.

40. The bronchoscope of claim 38, further comprising a second non-inflatable expandable member coupled to an outer surface of the distal end portion of the shaft, the second expandable member being longitudinally spaced from the first expandable member such that the needle port is interposed between the first expandable member and the second expandable member.

41. The bronchoscope of claim 38, wherein the needle and the expandable member are each formed of a shape-memory material.

42. The bronchoscope of claim 41, wherein the shape-memory material is a nickel-titanium alloy.

43. The bronchoscope of claim 38, wherein the needle is a biopsy needle.

44. The bronchoscope of claim 38, wherein the elongated tubular shaft is rigid.
